(19) United States
(12) Patent Application Publication
(10) Pub. No.: US 2014/0323745 A1
(43) Pub. Date: Oct. 30, 2014

(54) Polyphenolic selenium compound having functional group of alkali metal ion and selenium complex

(71) Applicant: Shanghai Ai Qi Ecological Technology Co., Ltd., Shanghai (CN)

(72) Inventors: Gong Chen, Shanghai (CN); Kunyuan Song, Shanghai (CN)

(73) Assignee: Shanghai Ai Qi Ecological Technology Co., Ltd., Shanghai (CN)

(21) Appl. No.: 14/158,819

(22) Filed: Jan. 18, 2014

Related U.S. Application Data
(63) Continuation-in-part of application No. PCT/CN2013/078719, filed on Jul. 3, 2013.

(30) Foreign Application Priority Data
Apr. 25, 2013 (CN) 201310147738.6

(51) Int. Cl. C07D 329/00; C07D 311/32; C07C 61/13

(52) U.S. Cl. CPC C07D 329/00; C07C 61/13; C07D 311/32
USPC 549/400; 562/502

(57) ABSTRACT

A polyphenolic selenium compound having a functional group of alkali metal ion and selenium complex which has an aromatic ring is provided, wherein the aromatic ring comprises at least two functional groups, each of which is one member selected from the group consisting of oxygen functional group, sulphur functional group, phosphorus functional group and nitrogen functional group; and selenium complex functional group is formed by selenium, alkali metal ion and the functional group. The polyphenolic selenium compound having a functional group of alkali metal ion and selenium complex of the present invention has characteristics of over 20% selenium content and no toxicity. Because it has revolutionary effects in killing bacteria, virus and cancer cell, enhancing human immunity, removing oxygen free radicals and etc., it holds a vital medicinal value.

### CROSS REFERENCE OF RELATED APPLICATION

This is a Continuation-In-Parts application of an application having an application number PCT/CN2013/078719, filed Jul. 3, 2013, which claims priority under 35 U.S.C. 119(a-d) to CN 201310147738.6, filed Apr. 25, 2013.

### BACKGROUND OF THE PRESENT INVENTION

1. Field of Invention

The present invention relates to a new type organic selenium compound, and more particularly to a polyphenolic selenium compound having a functional group of alkali metal ion and selenium complex.

2. Description of Related Arts

Selenium is an essential trace element for human metabolism. However, the uneven distribution of selenium element on the earth causes that over 40 countries and regions are deficient in selenium, and long-term residences thereof are more likely to be infested with diseases such as cardio-cerebrovascular disease, liver disease, diabetes mellitus and cancer. Thus, the long-term residences thereof need selenium supplements. Due to high toxicity and indigestibility thereof, the inorganic selenium has been forbidden in some countries. The commercially available organic selenium has characteristics of low toxicity and high bioavailability, but has low selenium and side effects of accumulating in human body. Therefore, how to develop optimum selenium source has been a research focus. European and American professionals have already realized that in order to treat serious diseases, the organic selenium must be in an ultra high dose. However, organic selenium which is in an ultra high dose and nontoxic does not exist.

A Chinese patent with a publication number CN20111035685 Preparation technology for complex of zinc and tea polyphenol and a Chinese patent with a publication number CN201010259488 Ruthenium-selenium complex and method thereof in preparing fluorescence probe and antitumor drugs both disclosed a complex formed by selenium and metal. However, both zinc and ruthenium are not alkali metal. Furthermore, the ruthenium is heavy metal which belongs to cytotoxicity material.

A Chinese patent with a publication number CN200710066974 discloses a preparing method for complex of o-dihydroxy flavone and selenium and medical utilization thereof. The o-dihydroxy flavone is polyphenolic substance and is capable of reacting with selenium dioxide, wherein selenium is inserted between two oxygen atoms, H is removed, and obtained complex has structures as follows:

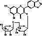

However, o-dihydroxy flavone is not capable of reacting with alkali metal ion, and mother nucleus thereof is easy to be destroyed to cause denaturation.

### SUMMARY OF THE PRESENT INVENTION

An object of the present invention is to provide a new type non-toxic organic selenium compound: polyphenolic selenium compound having a functional group of alkali metal ion and selenium complex.

Another object of the present invention is to provide a non-toxic organic selenium compound with high selenium content.

Another object of the present invention is to provide an organic selenium compound with therapeutic effects on various diseases.

Accordingly, in order to accomplish the above objects, the present invention provides a polyphenolic selenium compound having a functional group of alkali metal ion and selenium complex which has an aromatic ring, wherein:

the aromatic ring comprises at least two functional groups, each of which is one member selected from the group consisting of oxygen functional group, sulphur functional group, phosphorus functional group and nitrogen functional group; and a selenium complex functional group is formed by selenium, alkali metal ion and the functional group.

Beneficial effects of the present invention are described as follows. The polyphenolic selenium compound having a functional group of alkali metal ion and selenium complex of the present invention has characteristics of over 20% selenium content and no toxicity, and has revolutionary effects in killing bacteria, virus and cancer cell, enhancing human immunity, removing oxygen free radicals and etc.

These and other objectives, features, and advantages of the present invention will become apparent from the following detailed description, the attached illustrations, and the appended claims.

### BRIEF DESCRIPTION OF THE DRAWINGS

FIG. 1 is a high performance liquid chromatography diagram of a polyphenolic selenium compound having a functional groups of alkali metal ion and selenium complex obtained according to an example 3 of the present invention, wherein curve 1 is a high performance liquid chromatography diagram of a polyphenolic selenium compound having a functional group of potassium ion and selenium complex, and curve 2 is a polyphenolic selenium compound having a functional group of sodium ion and selenium complex.

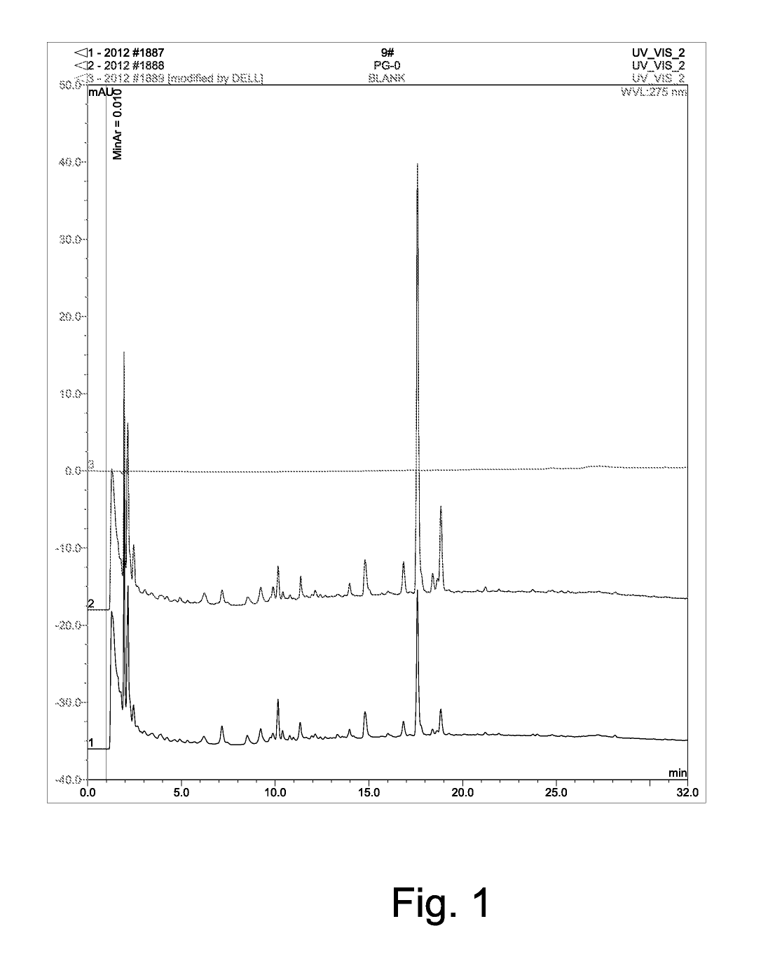

FIG. 2 is a high performance liquid chromatography diagram of the polyphenolic selenium compound having functional groups of alkali metal ion and selenium complex obtained according to an example 1 of the present invention.

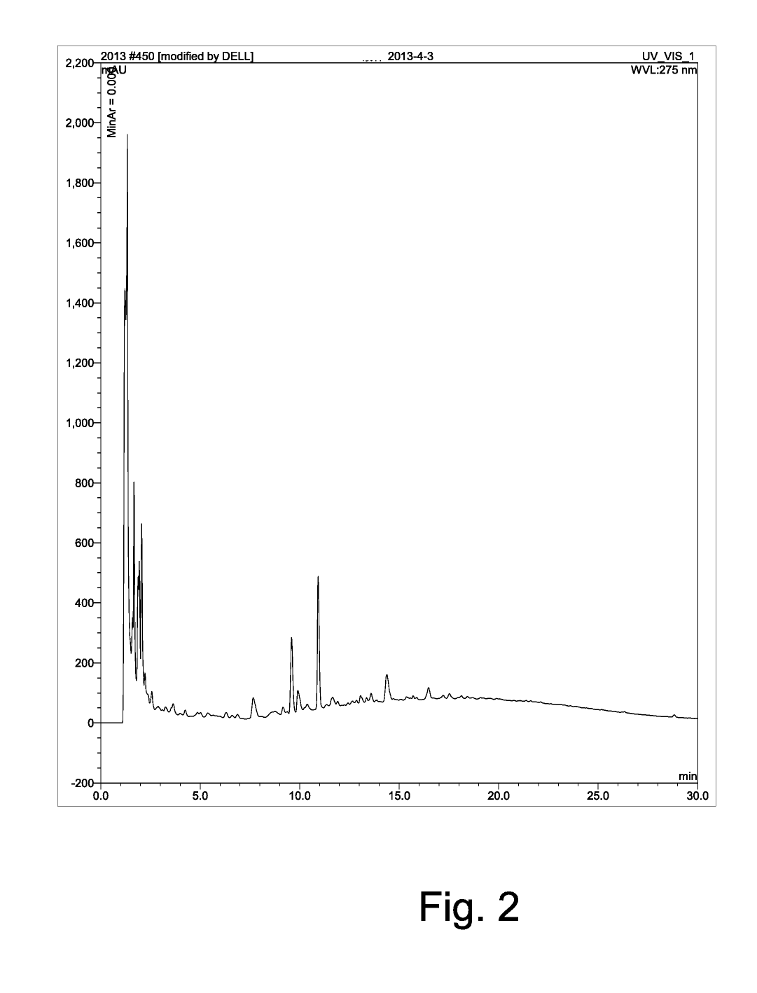

### DETAILED DESCRIPTION OF THE PREFERRED EMBODIMENT

According to a preferred embodiment of the present invention, a polyphenolic selenium compound having a functional group of alkali metal ion and selenium complex is illustrated, wherein:

the aromatic ring comprises at least two functional groups, each of which is one member selected from the group consisting of oxygen functional group, sulphur functional group, phosphorus functional group and nitrogenfunctional group; and selenium complex functional group is formed by selenium, alkali metal ion and the functional group.

According to a preferred embodiment of the present invention, the polyphenolic selenium compound having a functional group of alkali metal ion and selenium complex is illustrated, wherein the oxygen functional group comprises: hydroxyl, carboxylic group, phenolic group, quinonyl, quinonyl and hydroxyl, alcoholic hydroxyl, phenolic hydroxyl, sulfonic group, amino group, free quinonyl, semiquinone, quinonic oxygen group, monomethyl, and at least one kind monomethyl-active functional group which comprises methoxyl, carboxymethyl, hydroxymethyl, phenolic methyl and methylamino group.

According to a preferred embodiment of the present invention, the polyphenolic selenium compound having a functional group of alkali metal ion and selenium complex comprises following structure of:

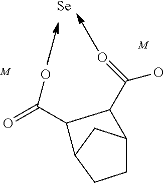

wherein M is alkali metal ion.

According to a preferred embodiment of the present invention, the polyphenolic selenium compound having a functional group of alkali metal ion and selenium complex comprises following structure of:

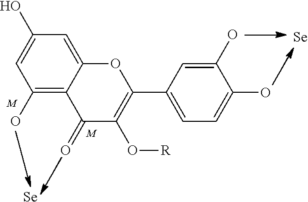

wherein R═CH₃, CH₂CH₂CH₃.

According to a preferred embodiment of the present invention, the polyphenolic selenium compound having a functional group of alkali metal ion and selenium complex comprises following structure of:

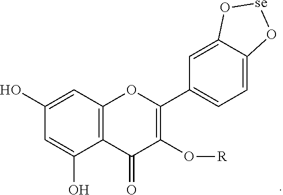

wherein R is alkali metal ion and selenium complex.

According to a preferred embodiment of the present invention, the polyphenolic selenium compound having a functional group of alkali metal ion and selenium complex is illustrated, wherein R has following structure of:

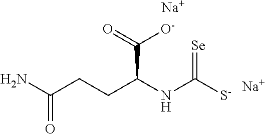 [SMILES: [Na+] ; NC(=O)CC[C@H](NC([S-])=[Se])C(=O)[O-] ; [Na+]]

According to a preferred embodiment of the present invention, the polyphenolic selenium compound having a functional group of alkali metal ion and selenium complex is illustrated, wherein R has following structure of:

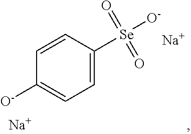 [SMILES: O=[Se](=O)([O-])c1ccc([O-])cc1 ; [Na+] ; [Na+]]

According to a preferred embodiment of the present invention, the polyphenolic selenium compound having a functional group of alkali metal ion and selenium complex is illustrated, wherein R has following structure of:

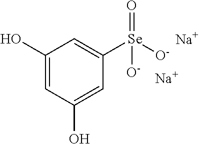

According to a preferred embodiment of the present invention, the polyphenolic selenium compound having a functional group of alkali metal ion and selenium complex is illustrated, wherein R has following structure of:

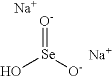

According to a preferred embodiment of the present invention, the polyphenolic selenium compound having a functional group of alkali metal ion and selenium complex is illustrated, wherein R has following structure of:

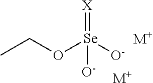

wherein M is alkali metal ion, X is N, S or P.

According to a preferred embodiment of the present invention, the polyphenolic selenium compound having a functional group of alkali metal ion and selenium complex is illustrated, wherein its molecular weight thereof is 100˜600.

According to a preferred embodiment of the present invention, aqueous solution of the polyphenolic selenium compound having a functional group of alkali metal ion and selenium complex is weakly alkaline, pH thereof is 7.2˜8.5, water-solubility thereof is high, and lipophilicity thereof is good.

According to a preferred embodiment of the present invention, a preparing process of the polyphenolic selenium compound having a functional group of alkali metal ion and selenium complex comprises following steps of:

1. obtaining one kind of multiple-structural polyphenolic compound by means of biotechnological hydrolysis, wherein the multiple-structural polyphenolic compound is weakly acidic (pH: 4.5˜6.5), and has good water-solubility, wherein:

molecules of the multiple-structural polyphenolic compound have aromatic rings or other heterocycles such as pyrrole, furan, indole and etc.; the aromatic rings are connected by bridge bond; the aromatic rings have a variety of active functional groups comprising: hydroxyl, carboxylic group, phenolic group, phenolic hydroxyl, quinonyl, quinonyl and hydroxyl, alcoholic hydroxyl, sulfonic group, amino group, free quinonyl, semiquinone, quinonic oxygen group, monomethyl, and at least one kind monomethyl-active functional group which comprises methoxyl, carboxymethyl, hydroxymethyl, phenolic methyl and methylamino group;

2. reacting the multiple-structural polyphenolic compound with at least one kind of inorganic alkali metal to obtain low-aromaticity multivalent phenolic hydroxyl carboxylate, which is polymeric, nonhomogeneous, alkaline (pH: 10˜12), has high solubility and is capable of dissolving into multiple solvents;

3. reacting the multivalent phenolic hydroxyl carboxylate with SeO₂ to obtain low-aromaticity multivalent phenolic hydroxyl carboxylic acid selenium complex salts, wherein a functional group thereof is alkali metal ion and selenium complex, aqueous solution thereof is weakly alkaline (pH: 7.2˜8.0), water solubility thereof is high, and lipophilicity thereof is good;

wherein the multivalent phenolic hydroxyl carboxylic acid selenium complex salts consist of a plurality of polyphenolic structures with functional fragments of alkali metal ion and selenium complex.

Fundamental structure of the polyphenolic selenium compound having a functional group of alkali metal ion and selenium complex is bigeminal or poly-phenolic hydroxyl, methoxyl, carboxylic group, quinonyl and hydroxyl and etc.

The polyphenolic selenium compound having the functional group of alkali metal ion and selenium complex is new produced compound.

Principle of the present invention is as follows. Taking advantage of isosteric principle, N, S or P in functional groups of the multiple-structural polyphenolic compounds is replaced by Se, or N, S or P in the functional groups of the multiple-structural polyphenolic compounds is connected with Se by covalent bond to form the alkali metal ion and selenium complex.

The alkali metal ion forms bidentate or multidentate coordinate bond with O, S, N or P, and O also forms bidentate or multidentate coordinate bond with Se.

### Example 1

A polyphenolic selenium compound having a functional group of alkali metal ion and selenium complex has the following structure of:

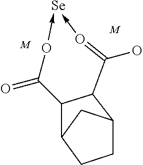

wherein M is alkali metal ion.

In this example, the structure

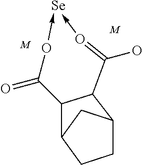

is capable of serving as a functional group

R in other structures.

In this example, a preparing process of the polyphenolic selenium compound having the functional group of alkali metal ion and selenium complex comprises following steps of:

a) adding 2.0% urea into lignosulfonate-water solution containing 20% solid formation for serving as growth medium (pH=6.0), wherein the lignosulfonate-water solution is extracted from depickling paper pulp by sulphuric acid; inoculating the growth medium with 2% mixed strains comprising: candida tropicalis, pseudomonas, candida utilis and strains of effective microorganisms from Japan, and fermenting for 72 hours under a temperature of 30° C. to obtain the multiple-structural polyphenolic compounds, wherein an inoculation proportion thereof is 1:2:2:2; and

b) reacting the multiple-structural polyphenolic compounds with sodium hydroxide to obtain multivalent phenolic hydroxyl sodium carboxylate, wherein multiple-structural polyphenolic compounds:sodium hydroxide=1:1˜0.1, wherein a reaction temperature thereof is 120° C., and materials are mechanically stirred to be uniformly mixed while reacting; and

c) reacting the multivalent phenolic hydroxyl sodium carboxylate with SeO₂ to obtain multivalent phenolic hydroxyl carboxylic acid selenium complex salts, wherein the multivalent phenolic hydroxyl carboxylic acid selenium complex salts are organic selenium composition, multivalent phenolic hydroxyl sodium carboxylate:SeO₂=1:1˜0.1, a reaction temperature thereof is 200° C., and materials are mechanically stirred to be uniformly mixed while reacting.

Moreover, other various structures of compounds of the present invention are also obtained by means of the above mentioned preparing process of the polyphenolic selenium compound having the functional group of alkali metal ion and selenium complex.

### Example 2

A polyphenolic selenium compound having a functional group of alkali metal ion and selenium complex has the following structure of:

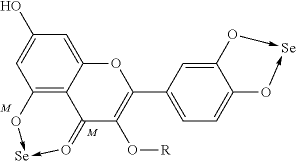

wherein R═CH₃, CH₂CH₂CH₃.

In this example, a preparing process of the polyphenolic selenium compound having the functional group of alkali metal ion and selenium complex comprises following steps of:

a) adding 2.0% urea into lignosulfonate-water solution containing 20% solid formation for serving as growth medium (pH=6.0), wherein the lignosulfonate-water solution are extracted from depickling paper pulp by sulphuric acid; inoculating the growth medium with 2% mixed strains comprising: candida tropicalis, pseudomonas, candida utilis and strains of effective microorganisms from Japan, and fermenting for 72 hours under a temperature of 30° C. to obtain the multiple-structural polyphenolic compounds, wherein an inoculation proportion thereof is 1:2:2:2; and

b) reacting the multiple-structural polyphenolic compounds with potassium hydroxide to obtain multivalent phenolic hydroxyl potassium carboxylate, wherein multiple-structural polyphenolic compounds: potassium hydroxide=1:1˜0.1, wherein a reaction temperature thereof is 120° C., and materials are mechanically stirred to be uniformly mixed while reacting; and

c) reacting the multivalent phenolic hydroxyl potassium carboxylate with SeO₂ to obtain multivalent phenolic hydroxyl carboxylic acid selenium complex salts, wherein the multivalent phenolic hydroxyl carboxylic acid selenium complex salts are organic selenium composition, multivalent phenolic hydroxyl potassium carboxylate: SeO₂=1:1˜0.1, a reaction temperature thereof is 200° C., and materials are mechanically stirred to be uniformly mixed while reacting.

Moreover, other various structures of compounds of the present invention are also obtained by means of the above mentioned preparing process of the polyphenolic selenium compound having the functional group of alkali metal ion and selenium complex.

### Example 3

A polyphenolic selenium compound having a functional group of alkali metal ion and selenium complex has the following structure of:

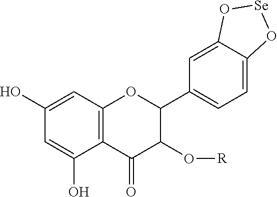

wherein R is alkali metal ion.

In this example, a preparing process of the polyphenolic selenium compound having the functional group of alkali metal ion and selenium complex comprises following steps of:

a) adding 2.0% urea into lignosulfonate-water solution containing 20% solid formation for serving as growth medium (pH=6.0), wherein the lignosulfonate-water solution are extracted from depickling paper pulp by sulphuric acid; inoculating the growth medium with 2% mixed strains comprising: candida tropicalis, pseudomonas, candida utilis and strains of effective microorganisms from Japan, and fermenting for 72 hours under a temperature of 30° C. to obtain the multiple-structural polyphenolic compounds, wherein an inoculation proportion thereof is 1:2:2:2; and

b) reacting the multiple-structural polyphenolic compounds with at least one kind of inorganic metal base such as NaOH or KOH to obtain multivalent phenolic hydroxyl sodium carboxylate or multivalent phenolic hydroxyl potassium carboxylate, etc.; and

c) reacting the multivalent phenolic hydroxyl sodium/potassium carboxylate and etc. with SeO₂ to obtain multivalent phenolic hydroxyl carboxylic acid selenium complex salts comprising Na or K or other alkali metal, wherein the multivalent phenolic hydroxyl carboxylic acid selenium complex salts are organic selenium composition, multivalent phenolic hydroxyl carboxylic acid selenium complex salts comprising alkali metal: SeO₂=1:1˜0.1, a reaction temperature thereof is 200° C., and materials are mechanically stirred to be uniformly mixed while reacting.

Moreover, other various structures of compounds of the present invention are also obtained by means of the above mentioned preparing process of the polyphenolic selenium compound having the functional group of alkali metal ion and selenium complex.

One skilled in the art will understand that the embodiment of the present invention as shown in the illustrations and described above is exemplary only and not intended to be limited.

It will thus be seen that the objects of the present invention have been fully and effectively accomplished. Its embodiments have been shown and described for the purposes of illustrating the functional and structural principles of the present invention and are subject to change without departure from such principles. Therefore, this invention includes all modifications encompassed within the spirit and scope of the following claims.

In following toxicity test, sample-1, sample-2 and sample-3 are multivalent phenolic hydroxyl carboxylate produced according to an embodiment 1, an embodiment 2 and an embodiment 3 of the present invention, respectively.

### Acute Toxicity Test of Sample-1 in Mice by Intragastrical Administration

### Summary

In this test, acute toxicity of sample-1 in mice by one-time intragastrical administration is observed. Poisoning manifestations and death rate of the mice tested is observed within 14 days of poisoning. Results show that LD₅₀ of the sample-1 in the mice by intragastrical administration is 260 mg·kg⁻¹.

1 Object: observing acute toxicity reaction and death rate of the mice by intragastrical administration of the sample-1.

2 Medicine for testing and menstruum

2.1 The medicine for testing

Name: sample-1

Shape and properties: brown powder

Content: 208 mg/g

Batch number: 20120611

2.2 The menstruum: 0.5% sodium carboxy methyl cellulose (CMC)

3. Animals

Source: Kunming mice, provided by Department of Animal Science, Fudan University.

Certification number: SCXK (Shanghai) 2009-0019

Weight: 18˜22 g

Sexuality: half male and half female

4. Externally environmental conditions for feeding and administrating the animals

4.1 Environmental conditions: temperature: 18˜22° C., humidity: 45˜65%

4.2 Feeding facility: SPF laboratory animal house

5 Feed: mice feed, provided by Shanghai Shilin Biological Science and Technology Ltd. and a batch number thereof is Q/TJCX 1-2010.

6 Bedding: wood shavings, provided by Department of Animal Science, Fudan University.

7 Information about drinking water: tap water.

8 Experimental method

8.1 Designed dose rate: 4 groups are divided in this experiment, and the dose rates thereof are 490 mg/kg, 343 mg/kg, 240 mg/kg and 168 mg/kg, respectively; and a group interval thereof is 0.7.

8.2 Preparation method of the medicine for testing (the sample-1): adding 0.5% CMC (Carboxy Methylated Cellulose) to the sample-1 and grinding it, then successively diluting it to suspensions of corresponding density.

8.3 Medicine administration methods and approaches: The animals are randomly divided into 4 groups according to their sexuality, and each group has 10 mice, with half male and half female. All of the mice are fasted for 14 hours (supplying nothing but water) before experiments. Then the mice are administered intragastrically with the medicine by a dose of 20 ml/kg. Poisoning and death conditions of the animals are observed within 14 days after the medicine administration.

9 Operation, inspection and determination of the experiment

9.1 General symptoms: observing poisoning symptom of the mice in activity behaviors, fur and excreta, and observing lethality.

9.2 Weight determination: weighing the mice once before the medicine administration, one week after the beginning of the experiment and before executing the mice when the experiment is over, respectively.

9.3 Observation indexes: processing gross anatomy on animals dead during the course of the experiment and animals still alive when the experiment is over, observing pathologic changes of principal organs by naked eye.

9.4 Observation period: observing the animals every 10 minutes within 4 hours after the animals are poisoned, and then observing the animals twice a day, morning and afternoon, respectively.

10 Data processing: LD₅₀ and 95% confidence limit are calculated by processing the data with SPSS16.0 statistical software using weighted probit regression model (Bliss method).

11 Results: In high-dose group, spontaneous activity of the mice decreases. After about 30 minutes the mice start to lie in the prone position, coating hair thereof tarnishes, and mild diarrhea appears on part of the mice. After being poisoned for 1 hour, the mice begin to die, and the death mainly appears within 4 hours. Dying mice have a symptom of clonic convulsion. Poisoned mice return to normal in 3 days. In mid-dose and low-dose groups, toxicity reaction of the mice caused by poisoning is the same as that in the high-dose group, but the only difference lies in a delay of poisoning time. After the experiment, all of the mice gain weight to varying degrees. (See Table 1)

After processing gross anatomy on mice dead in the experiment and mice still alive when the experiment is over, no obvious pathological changes demonstrate by observing with naked eye.

Referring to Table 2 for LD₅₀ and 95% confidence limit of sample-1 in the mice by intragastrical administration.

TABLE 1
Effect of acute toxicity of the sample-1 by intragastrical
administration on weight changes of the mice ( x̅ ± s)

| | | | |
|---|---|---|---|
| Dosage | | | |
| (mg · kg⁻¹) | 0 d | 7 d | 14 d |

490
343
240
168

TABLE 2
LD₅₀ and95% confidence limit of sample-1
in mice by intragastrical administration

| | | | | Death | LD₅₀ and |
|---|---|---|---|---|---|
| Dosage | Dosage | Animal | Death | rate | 95% confidence |
| (mg · kg⁻¹) | Logarithm | count | count | (%) | limit |
| 490 | 2.690 | 10 | 9 | 90 | LD₅₀ = 260 mg · kg⁻¹ |
| 343 | 2.535 | 10 | 6 | 60 | 95% confidence |
| 240 | 2.380 | 10 | 4 | 40 | limit is |
| 168 | 2.225 | 10 | 3 | 30 | 167~357 mg · kg⁻¹ |

12 Conclusion

LD₅₀ of the sample-1 is 260 mg/kg, and 95% confidence limit thereof is 167˜357 mg/kg.

### REFERENCES

1. Guiding principle for investigative techniques of acute toxicity of chemical medicine, guiding principle number [H] GPT 1-1
2. General principle for non-clinical safety technical evaluation of therapeutic biologics, guiding principle number [S] GTP 1-1

### Acute Toxicity Test of Sample-2 in Mice by Intragastrical Administration

### Summary

In this test, acute toxicity of sample-2 in mice by one-time intragastrical administration is observed. Poisoning manifestations and death rate of the mice tested is observed within 14 days of poisoning. Results show that LD₅₀ of the sample-2 in the mice by intragastrical administration is 328 mg·kg⁻¹.

1 Object: observing acute toxicity reaction and death rate of the mice by intragastrical administration of the sample-2.

2 Medicine for testing and menstruum

2.1 The medicine for testing

Name: sample-2

Shape and properties: brown powder

Content: 208 mg/g

Batch number: 20120618

2.2 The menstruum: 0.5% sodium carboxy methyl cellulose (CMC)

3. Animals

Source: Kunming mice, provided by Department of Animal Science, Fudan University.

Certification number: SCXK (Shanghai) 2009-0019

Weight: 18˜22 g

Sexuality: half male and half female

4. Externally environmental conditions for feeding and administrating the animals

4.1 Environmental conditions: temperature: 18˜22° C., humidity: 45˜65%

4.2 Feeding facility: SPF laboratory animal house

5 Feed: mice feed, provided by Shanghai Shilin Biological Science and Technology Ltd. and a batch number thereof is Q/TJCX 1-2010.

6 Bedding: wood shaving, provided by Department of Animal Science, Fudan University.

7 Information about drinking water: tap water.

8 Experimental method

8.1 Designed dose rate: 5 groups are divided in this experiment, and the dose rates thereof are 600 mg/kg, 450 mg/kg, 338 mg/kg, 253 mg/kg and 190 mg/kg respectively; and a group interval thereof is 0.75.

8.2 Preparation method of the medicine for testing (thesample-2): adding 0.5% CMC (Carboxy Methylated Cellulose) to the sample-2 and grinding it, then successively diluting it to suspensions of corresponding density.

8.3 Medicine administration methods and approaches: The animals are randomly divided into 5 groups according to their sexuality, and each group has 10 mice, with half male and half female. All of the mice are fasted for 14 hours (supplying nothing but water) before experiments. Then the mice are administered intragastrically with the medicine by a dose of 20 ml/kg. Poisoning and death conditions of the animals are observed within 14 days after the medicine administration.

9 Operation, inspection and determination of the experiment

9.1 General symptoms: observing poisoning symptom of the mice in activity behaviors, fur and excreta, and observing lethality.

9.2 Weight determination: weighing the mice once before the medicine administration, one week after beginning the experiment and before executing the mice when the experiment is over, respectively.

9.3 Observation indexes: processing gross anatomy on animals dead during the course of the experiment and animals still alive when the experiment is over, observing pathologic changes of principal organs by naked eye.

9.4 Observation period: observing the animals every 10 minutes within 4 hours after the animals are poisoned, and then observing the animals twice a day, morning and afternoon, respectively.

10 Data processing: LD₅₀ and 95% confidence limit are calculated by processing the data with SPSS16.0 statistical software using weighted probit regression model (Bliss method).

11 Results: In high-dose group, spontaneous activity of the mice decreases. After about 15 minutes the mice start to lie in the prone position, coating hair thereof tarnishes, and mild diarrhea appears on part of the mice. After being poisoned for 30 minutes, the mice begin to die, and the death mainly appears in 4 hours. Dying mice have a symptom of clonic convulsion. Poisoned mice return to normal in 3 days. In mid-dose and low-dose groups, toxicity reaction of the mice caused by poisoning is the same as that in the high-dose group, but the only difference lies in a delay of poisoning time. After the experiment, all of the mice gain weight to varying degrees. (See Table 1)

After processing gross anatomy on mice dead in the experiment and mice still alive when the experiment is over, no obvious pathological changes demonstrate by observing with naked eye.

Referring to Table 2 for LD₅₀ and 95% confidence limit of sample-2 in the mice by intragastrical administration.

TABLE 1
Effect of acute toxicity of the sample-2 by intragastrical
administration on weight changes of the mice ( x̅ ± s)

| | | | |
|---|---|---|---|
| Dosage | | | |
| (mg · kg⁻¹) | 0 d | 7 d | 14 d |

600
450
338
253
190

TABLE 2
LD₅₀ and95% confidence limit of sample-2
in mice by intragastrical administration

| | | | | Death | LD₅₀ and |
|---|---|---|---|---|---|
| Dosage | Dosage | Animal | Death | rate | 95% confidence |
| (mg · kg⁻¹) | Logarithm | count | count | (%) | limit |
| 600 | 2.778 | 10 | 9 | 90 | LD₅₀ = 328 mg · kg⁻¹ |
| 450 | 2.653 | 10 | 7 | 70 | 95% confidence |
| 338 | 2.529 | 10 | 6 | 60 | limit is |
| 253 | 2.403 | 10 | 3 | 30 | 262~404 mg · kg⁻¹ |
| 190 | 2.225 | 10 | 1 | 10 | |

12 Conclusion

LD₅₀ of the sample-2 is 328 mg/kg, and 95% confidence limit thereof is 262˜404 mg/kg.

### REFERENCES

1. Guiding principle for investigative techniques of acute toxicity of chemical medicine, guiding principle number [H] GPT 1-1
2. General principle for non-clinical safety technical evaluation of therapeutic biologics, guiding principle number [S] GTP 1-1

### Acute Toxicity Test of Sample-3M Mice by Intragastrical Administration

### Summary

In this test, acute toxicity of the sample-3 in mice by one-time intragastrical administration is observed. Poisoning manifestations and death rate of the mice tested is observed within 14 days of poisoning. Results show that LD₅₀ of the sample-3 in the mice by intragastrical administration is 177 mg·kg⁻¹.

1 Object: observing acute toxicity reaction and death rate of the mice by intragastrical administration of the sample-3.

2 Medicine for testing and menstruum

2.1 The medicine for testing

Name: sample-3

Shape and properties: brown powder

Content: 104 mg/g

Batch number: 20120618

2.2 The menstruum: 0.5% sodium carboxy methyl cellulose (CMC)

3. Animals

Source: Kunming mice, provided by Department of Animal Science, Fudan University.

Certification number: SCXK (Shanghai) 2009-0019

Weight: 18˜22 g

Sexuality: half male and half female

4. Externally environmental conditions for feeding and administrating the animals

4.1 Environmental conditions: temperature: 18˜22° C., humidity: 45˜65%

4.2 Feeding facility: SPF laboratory animal house

5 Feed: mice feed, provided by Shanghai Shilin Biological Science and Technology Ltd. and a batch number thereof is Q/TJCX 1-2010.

6 Bedding: wood shaving, provided by Department of Animal Science, Fudan University.

7 Information about drinking water: tap water.

8 Experimental method

8.1 Designed dose rate: 4 groups are divided in this experiment, and the dose rates thereof are 253 mg/kg, 189 mg/kg, 142 mg/kg and 107 mg/kg, respectively; and a group interval thereof is 0.75.

8.2 Preparation method of the medicine for testing (thesample-3): adding 0.5% CMC (Carboxy Methylated Cellulose) to the sample-3 and grinding it, and then successively diluting it to suspensions of corresponding density.

8.3 Medicine administration methods and approaches: The animals are randomly divided into 4 groups according to their sexuality, and each group has 10 mice, with half male and half female. All of the mice are fasted for 14 hours (supplying nothing but water) before experiments. Then the mice are administered intragastrically with the medicine by a dose of 20 ml/kg. Poisoning and death conditions of the animals are observed within 14 days after the medicine administration.

9 Operation, inspection and determination of the experiment

9.1 General symptoms: observing poisoning symptom of the mice in activity behaviors, fur and excreta, and observing lethality.

9.2 Weight determination: weighing the mice once before the medicine administration, one week after beginning the experiment and before executing the mice when the experiment is over respectively.

9.3 Observation indexes: processing gross anatomy on animals dead during the course of the experiment and animals still alive when the experiment is over, observing pathologic changes of principal organs by naked eye.

9.4 Observation period: observing the animals every 10 minutes within 4 hours after the animals are poisoned, and then observing the animals twice a day, morning and afternoon, respectively.

10 Data processing: LD₅₀ and 95% confidence limit are calculated by processing the data with SPSS16.0 statistical software using weighted probit regression model (Bliss method).

11 Results: In high-dose group, spontaneous activity of the mice decreases. After about 30 minutes the mice start to lie in the prone position, coating hair thereof tarnishes, and mild diarrhea appears on part of the mice. After being poisoned for 1 hour, the mice begin to die, and the death mainly appears in 4 hours. Dying mice have a symptom of clonic convulsion. Poisoned mice return to normal in 3 days. In mid-dose and low-dose groups, toxicity reaction of the mice caused by poisoning is the same as that in the high-dose group, but the only difference lies in a delay of poisoning time. After the experiment, all of the mice gain weight to varying degrees. (See Table 1)

After processing gross anatomy on mice dead in the experiment and mice still alive when the experiment is over, no obvious pathological changes demonstrate by observing with naked eye.

Referring to Table 2 for LD₅₀ and 95% confidence limit of sample-3 in the mice by intragastrical administration.

TABLE 1
Effect of acute toxicity of the sample-3 by intragastrical
administration on weight changes of the mice ( x̅ ± s)

| | | | |
|---|---|---|---|
| Dosage | | | |
| (mg · kg⁻¹) | 0 d | 7 d | 14 d |

253
189
142
107

TABLE 2
LD₅₀ and95% confidence limit of sample-3
in mice by intragastrical administration

| | | | | Death | LD₅₀ and |
|---|---|---|---|---|---|
| Dosage | Dosage | Animal | Death | rate | 95% confidence |
| (mg · kg⁻¹) | Logarithm | count | count | (%) | limit |
| 253 | 2.403 | 10 | 8 | 80 | LD₅₀ = 177 mg · kg⁻¹ |
| 189 | 2.276 | 10 | 5 | 50 | 95% confidence |
| 142 | 2.152 | 10 | 3 | 30 | limit is |
| 107 | 2.029 | 10 | 2 | 20 | 137~256 mg · kg⁻¹ |

12 Conclusion

LD₅₀ of the sample-3 is 177 mg/kg, and 95% confidence limit thereof is 137˜256 mg/kg.

### REFERENCES

1. Guiding principle for investigative techniques of acute toxicity of chemical medicine, guiding principle number [H] GPT 1-1
2. General principle for non-clinical safety technical evaluation of therapeutic biologics, guiding principle number [S] GTP 1-1

According to an amount of an average LD₅₀ reported in the three acute toxicity tests of the mice mentioned above, if the organic selenium composition is applied to a 60-kilogram human body, a dosage of over 200000 μg per day is taken and no toxicity and side effects will be found. The dosage are 500˜1000 times of a dosage per day restricted by countries of the world (including China).

What is claimed is:

1. A polyphenolic selenium compound having a functional group of alkali metal ion and selenium complex which has an aromatic ring, wherein:
the aromatic ring comprises at least two functional groups,
each functional group is one member selected from the group consisting of oxygen functional group, sulphur functional group, phosphorus functional group and nitrogen functional group, and selenium complex functional group formed by selenium, alkali metal ion and the oxygen functional group, the sulphur functional group, the phosphorus functional group or the nitrogen functional group.

2. The polyphenolic selenium compound having a functional group of alkali metal ion and selenium complex, as recited in claim 1, wherein the oxygen functional group comprises: hydroxyl, carboxylic group, phenolic group, quinonyl, quinonyl and hydroxyl, alcoholic hydroxyl, phenolic hydroxyl, sulfonic group, amino group, free quinonyl, semiquinone, quinonic oxygen group, monomethyl, and at least one kind monomethyl-active functional group which comprises methoxyl, carboxymethyl, hydroxymethyl, phenolic methyl and methylamino group.

3. The polyphenolic selenium compound having a functional group of alkali metal ion and selenium complex, as recited in claim 1, wherein a structure thereof comprises 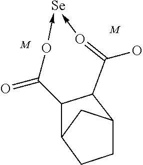
wherein M is alkali metal ion.

4. The polyphenolic selenium compound having a functional group of alkali metal ion and selenium complex, as recited in claim 1, wherein a structure thereof comprises: 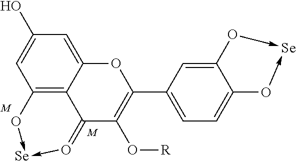
wherein R═CH₃, CH₂CH₂CH₃.

5. The polyphenolic selenium compound having a functional group of alkali metal ion and selenium complex, as recited in claim 1, wherein a structure thereof comprises: 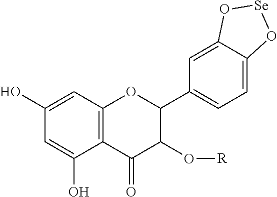
wherein R is alkali metal ion and selenium complex.

6. The polyphenolic selenium compound having a functional group of alkali metal ion and selenium complex, as recited in claim 5, wherein R has following structure of

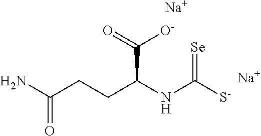 [SMILES: [Na+] ; NC(=O)CC[C@H](NC([S-])=[Se])C(=O)[O-] ; [Na+]]

7. The polyphenolic selenium compound having a functional group of alkali metal ion and selenium complex, as recited in claim 5, wherein R has following structure of

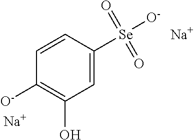 [SMILES: O=[Se](=O)([O-])c1ccc([O-])c(O)c1 ; [Na+] ; [Na+]]

8. The polyphenolic selenium compound having a functional group of alkali metal ion and selenium complex, as recited in claim 5, wherein R has following structure of

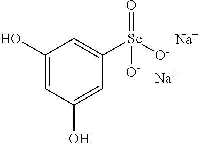

9. The polyphenolic selenium compound having a functional group of alkali metal ion and selenium complex, as recited in claim 5, wherein R has following structure of

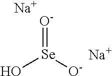

10. The polyphenolic selenium compound having a functional group of alkali metal ion and selenium complex, as recited in claim 5, wherein R has following structure of 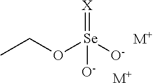
wherein M is alkali metal ion, X is N, S or P.

11. The polyphenolic selenium compound having a functional group of alkali metal ion and selenium complex, as recited in claim 1, wherein a molecular weight thereof is 100˜600.
